(19) United States
(12) Patent Application Publication
(10) Pub. No.: US 2012/0130204 A1
(43) Pub. Date: May 24, 2012

(54) SELF-CONTAINED PATIENT MONITOR

(76) Inventors: Joseph Charles Basta, Duvall, WA (US); Richard Lee Gagliardo, Sammamish, WA (US); Narayana Rathnakara, Miyapur (IN); Nityanand Shetty, Trimulgherry (IN); Robert Steurer, Sammamish, WA (US); William Oren Wekell, Maple Valley, WA (US)

(21) Appl. No.: 13/300,526

(22) Filed: Nov. 18, 2011

Related U.S. Application Data
(60) Provisional application No. 61/415,799, filed on Nov. 19, 2010.

(51) Int. Cl. A61B 5/0205; A61B 5/1455; A61B 5/01; A61B 5/0215; A61B 5/021; A61B 5/0402; A61B 5/083; A61B 5/145; A61B 5/0476; A61B 5/0488; A61B 5/087; A61B 5/00; A61B 5/02

(52) U.S. Cl. 600/301; 600/300; 600/483; 600/323; 600/549; 600/486; 600/485; 600/526; 600/532; 600/364; 600/544; 600/546; 600/538; 600/365; 600/509; 600/324

(57) ABSTRACT

A device for continuous, uninterrupted patient monitoring includes a portable, self-contained Patient Worn Hub (PWH) device. The PWH is a compact, lightweight patient monitoring device designed to remain with the patient for the duration of care. Parameter measurement devices connect to the PWH. Third party parameter measurement devices connect to the PWH via the use of a connection assembly that translates the information provided by the third party device to the protocol embedded within the PWH. The PWH is able to communicate with a bedside monitor via wired cables or wirelessly. Measured values are shown on external displays and/or on an optional integrated PWH touchscreen display. The PWH includes internal memory for storage of patient data and trends. The PWH optionally includes a docking station for providing operating and battery charging power. The PWH is assigned to each patient and will remain with that patient across multiple departments throughout the care process, thus eliminating the need for re-cabling patients.

### CROSS-REFERENCE TO RELATED APPLICATIONS

The present specification claims priority from U.S. Provisional Patent Application No. 61/415,799, entitled “Patient Monitoring System with Dual Serial Bus (DSB) Interface” and filed on Nov. 19, 2010, which is herein incorporated by reference in its entirety.

Co-pending U.S. patent application Ser. No. (to be determined), entitled “Dual Serial Bus Interface”, filed on Nov. 18, 2011 and assigned to the Applicant of the present invention, is also herein incorporated by reference in its entirety.

Co-pending U.S. patent application Ser. No. (to be determined), entitled “Configurable Patient Monitoring System”, filed on Nov. 18, 2011 and assigned to the applicant of the present invention, is also herein incorporated by reference in its entirety.

### FIELD

The present specification relates generally to patient monitoring systems. In particular, the present specification relates to a portable, wearable patient monitor for monitoring patient parameters. More particularly, the present specification relates to a self-contained patient monitor designed to remain with the patient for the duration of care that can communicate with both a bedside patient monitor and a patient monitoring network, either wired or wirelessly, and thus, continuously monitor the patient without interruption.

### BACKGROUND

A patient monitoring system is an electronic medical device that measures a patient's various vital signs, collects and processes all measurements as data, and then displays the data graphically and/or numerically on a viewing screen. Graphical data is displayed continuously as data channels on a time axis (waveforms). Patient monitoring systems are positioned near hospital beds, typically in critical care units, where they continually monitor patient status via measuring devices attached to the patient and can be viewed by hospital personnel. The systems are typically on a shelf, attached to the bed, or attached to a wall. Some patient monitoring systems can only be viewed on a local display, whereas others can be joined to a network and thereby display data at other locations, such as central monitoring or nurses' stations.

Portable patient monitoring systems are available for use by emergency medical services (EMS) personnel. These systems typically include a defibrillator along with the monitor. Other portable units, such as Holter monitors, are worn by patients for a particular time period and then returned to the physician for evaluation of the measured and collected data. Current patient monitoring systems are able to measure and display a variety of vital signs, including, pulse oximetry (SpO₂), electrocardiograph (ECG), invasive blood pressure (IBP), non-invasive blood pressure (NIBP), electroencephalograph (EEG), body temperature, cardiac output, capnography (CO₂), and respiration. Patient monitoring systems are capable of measuring and displaying maximum, minimum, and average values and frequencies, such as pulse and respiratory rates.

Data collected can be transmitted through fixed wire connections or wireless data communication. Power to patient monitoring systems can be supplied through a main power line or by batteries. While current patient monitoring systems are effective in monitoring patient conditions and notifying medical personnel of changes, they are not without certain drawbacks and limitations.

For example, conventional patient monitoring systems are generally large, bulky machines that cannot be transported easily. Such machines are difficult to use in ambulatory or mobile situations, resulting in temporary interruption of monitoring while the patient is being transferred from one location to another.

Therefore, a need exists for a lightweight portable, wearable patient monitoring device that continuously measures patient parameters and remains in communication with the hospital network. What is also needed is a portable device that is flexible in that it can readily adapt to changing acuity levels in various hospital environments and can operate in multiple modes.

Such a device will also require a uniform system for connecting parameter sensing devices. Mechanical enclosures meant for electrical medical devices need to be robust and meet regulatory requirements. Typical electrical medical devices include printed circuit boards (PCBs), connectors, cables and gaskets, which are all rigidly mounted inside the enclosure by means of mechanical components. Normally this may require three to four individual components to achieve. Multiple components call for a specific and careful assembly procedure, more inventories, and more maintenance and risk.

Further, devices used in medical applications have restrictions in material and process of manufacture and assembly. Hence, considering the complexities involved, manufacturers prefer conventional design and assembly methods. Also, electrical medical devices must be able to function reliably in hospital environments where the risk of water damage is high. Therefore, what is needed is a uniform adapter device which includes a mechanical enclosure that accepts a multitude of original equipment manufacturer (OEM) components that is simple to assemble and, in addition, prevents liquid ingress.

### SUMMARY

The present specification is directed toward a portable, self-contained device used to monitor patient parameters, comprising an internal battery, at least one connection for a parameter measuring instrument, and at least one connection assembly for connection of a third party measuring instrument, said connection assembly comprising printed circuit boards, a plug, and an enclosure, wherein said device: provides continuous, uninterrupted monitoring of at least one patient parameter; is capable of communicating with a bedside monitor and a patient monitoring network; is worn by and remains with an assigned patient for the duration of care; and, is designed in a compact and lightweight manner to enhance portability.

In one embodiment, the portable, self-contained device used to monitor patient parameters further comprises a connector for docking of said device within a docking station, wherein said docking station provides operating and battery charging power to said device, and wherein said docking station receives power from an external power supply.

In one embodiment, the patient parameters capable of being measured by the device include, but are not limited to, electrocardiograph (ECG)/respiration, pulse oximetry (SpO₂), temperature, invasive blood pressure (IBP), non-invasive blood pressure (NIBP), cardiac output, capnography, mixed venous oxygen saturation (SvO₂) and central venous oxygen saturation (SvcO₂), multi-gas analysis, bispectral index (BISx), neuromuscular transmission (NMT), transcutaneous oxygen (TcpO₂) & transcutaneous carbon dioxide (TcpCO₂), spirometry, blood glucose, pulmonary mechanics, electroencephalograph (EEG) and resultant entropy characteristics, auditory evoked potential (AEP), hemodialysis, hemofiltration, and 16-lead ECG.

In one embodiment, the portable, self-contained device used to monitor patient parameters further comprises an integrated touchscreen display.

In one embodiment, the portable, self-contained device used to monitor patient parameters further comprises alarm annunciation capabilities.

In one embodiment, the portable, self-contained device used to monitor patient parameters further comprises an integrated NIBP measuring instrument.

In one embodiment, the portable, self-contained device used to monitor patient parameters further comprises internal memory capable of storing measured patient parameter data. In one embodiment, the internal memory is capable of storing up to 24 hours of continuous patient data and up to 96 hours of trend data.

In one embodiment, the portable, self-contained device used to monitor patient parameters further comprises an internal real-time clock (RTC).

In one embodiment, the portable, self-contained device used to monitor patient parameters communicates through a wired connection with the bedside monitor via detachable cables. In another embodiment, the device comprises an internal wireless network card and is capable of communicating wirelessly with the bedside monitor, a parameter transceiver, and a hospital network.

In one embodiment, measured data is transmitted from the device to the bedside monitor and then presented in numerical and graphical form on an external display.

In one embodiment, the parameter measuring instrument is connected to the device via a Dual Serial Bus (DSB) connection.

In one embodiment, the portable, self-contained device used to monitor patient parameters further comprises a securing band or similar structure for securing the device to the patient during transport and ambulation.

In one embodiment, the portable, self-contained device used to monitor patient parameters comprises an internal battery, an integrated NIBP measuring instrument, an integrated SpO₂ measuring instrument, an integrated ECG/respiration instrument, at least one connection for a parameter measuring instrument, and at least one connection assembly for connection of a third party measuring instrument, said connection assembly comprising printed circuit boards, a plug, and an enclosure. In another embodiment, the device comprises the same components as the device described directly above, plus, an integrated touchscreen display, alarm annunciation, and limited data storage capability.

The present specification is also directed toward a connection assembly used to translate electronic information from a first protocol to a second protocol, comprising: a plug at a first end of said assembly for connecting a first device cable from a first device, said first device operating on said first protocol; at least one first connector at a second end of said assembly opposite said first end of said assembly, for connecting at least one printed circuit board; at least one second connector at said second end of said assembly opposite said first end of said assembly, for connecting at least one second device cable from a second device, said second device operating on said second protocol; at least one printed circuit board capable of translating said electronic information from said first protocol to said second protocol, said printed circuit board connected to said at least one first connector at said second end of said assembly; and, at least one movable elongate member attached to said plug along an exterior edge of said plug, said at least one movable elongate member movable from a first position to a second position wherein said movable elongate member forms an enclosure at least partially encompassing said at least one printed circuit board and said second device cable when said movable elongate member is in said second position.

In one embodiment, the plug includes a gasket around the outer edge of said plug to prevent liquid ingress into the assembly.

The present specification is also directed toward a system for providing continuous patient monitoring, comprising: a portable, self-contained device used to monitor patient parameters, comprising an internal battery, at least one connection for a parameter measuring instrument, and at least one connection assembly for connection of a third party measuring instrument, said connection assembly comprising printed circuit boards, a plug, and an enclosure, wherein; said device provides continuous, uninterrupted monitoring of at least one patient parameter; said device is capable of communicating with a bedside monitor and a patient monitoring network; said device is worn by and remains with an assigned patient for the duration of care; and, said device is designed in a compact and lightweight manner to enhance portability; at least one patient parameter measuring instrument connected to the device; a bedside monitor in communication with the device; at least one external display connected to the bedside monitor; and, a hospital network in communication with the bedside monitor.

In another embodiment, portable, self-contained device included in the system described above comprises an integrated wireless network card, wherein the device is capable of direct communication with the hospital network.

### BRIEF DESCRIPTION OF THE DRAWINGS

These and other objects and advantages will become more fully apparent from the following detailed description when read in conjunction with the accompanying drawings with like reference numerals indicating corresponding parts through-out, wherein:

FIG. 1 is an oblique front view illustration of one embodiment of the Patient Worn Hub (PWH) of the present invention;

FIG. 2 is an oblique front view illustration of another embodiment of the Patient Worn Hub (PWH) including an optional securing band;

FIG. 3A is an oblique front view illustration of one embodiment of the Patient Worn Hub docking unit of the present invention;

FIG. 3B is an oblique front view illustration of one embodiment of the Patient Worn Hub docking unit depicting the PWH placed into the docking unit;

FIG. 4 is an illustration of a multitude of possible embodiments of the Patient Worn Hub connected to a hospital network;

FIG. 5A is an oblique front view illustration of one embodiment of the hinge connector assembly in the open position prior to assembly;

FIG. 5B is a side view illustration of one embodiment of the hinge connector assembly in the open position prior to assembly;

FIG. 6 is an oblique front view illustration of one embodiment of the hinge connector assembly in the open position with a DSB device cable in place;

FIG. 7A is an oblique front view illustration of one embodiment of the hinge connector assembly fully assembled with a DSB device cable attached;

FIG. 7B is a side view illustration of one embodiment of the hinge connector assembly fully assembled with a DSB device cable attached;

FIG. 8 is a block diagram illustrating the components included in one embodiment of the Patient Worn Hub;

FIG. 9 is a block diagram illustrating one embodiment of the design of the electrical architecture of the PWH;

FIG. 10 is a block diagram illustrating one embodiment of an exemplary processor included in the electrical architecture of the PWH;

FIG. 11 is a block diagram illustrating one embodiment of an upstream USB port of the PWH; and,

FIG. 12 is a block diagram illustrating one embodiment of the power section of the PWH.

### DETAILED DESCRIPTION

The present specification is directed toward a Patient Worn Hub (PWH) device for monitoring patient parameters. The PWH is a self-contained patient monitoring device designed to remain with the patient for the duration of care. The PWH is assigned to each patient and will remain with that patient across multiple departments throughout the care process, thus eliminating the need for re-cabling patients. The PWH is designed in a compact and lightweight manner making it easily portable.

In one embodiment, the PWH is able to communicate with the bedside monitor of the patient monitoring system and provides continuous, uninterrupted patient monitoring. The PWH is capable of communicating with the monitor of the patient monitoring system described in co-pending U.S. patent application Ser. No. (to be determined), entitled “Configurable Patient Monitoring System”, filed on Nov. 18, 2011 and assigned to the Applicant of the present invention, which is hereby incorporated by reference. In one embodiment, the PWH communicates with the monitor via wired cables. In another embodiment, the PWH communicates with the monitor wirelessly, allowing the patient to be transferred to a different location while the monitor remains in the patient room.

The PWH connects to the monitor and the parameter measuring devices of a patient monitoring system via Dual Serial Bus (DSB) connectors and cables. The DSB interface comprises a first serial protocol and a second serial protocol, wherein the first protocol is a USB, Firewire, or Ethernet protocol and the second serial protocol is a Low Power Serial (LPS) protocol. Within the DSB interface, each component of the patient monitoring system is a DSB Host, DSB Device, or, both a DSB Host and DSB Device. A DSB Host is in communication with and can supply operating and battery charging power to a connected DSB Device and also contains a switched Auxiliary Voltage Supply (AVS) which can provide up to 15 W of power to attached DSB Devices for battery charging or other high power needs. The DSB interface is presented in greater detail in co-pending U.S. patent application Ser. No. (to be determined), entitled “Dual Serial Bus Interface”, filed on Nov. 18, 2011 and assigned to the Applicant of the present invention, which is hereby incorporated by reference.

The following terms and abbreviations are used throughout the specification:

“PWH” refers to Patient Worn Hub, which is a host that will run the algorithms needed for patient parameter processing, whereby the PWH may have several DSB Host interfaces. “PT” refers to a Parameter Transceiver, which is a wireless transceiver that will send patient parameter data to a central system for processing. In one embodiment, the PT has two DSB Host interfaces.

“DSB” is a Dual Serial Bus, an interface of the present invention that supports communication and power transfer via both USB and LPS protocols. The DSB interface includes at least six conductors which physically connect a DSB Host to a DSB Device. In one embodiment, the six conductors comprise: a virtual bus (VBUS) conductor for power transfer, two data transmission conductors, a ground conductor, an auxiliary voltage supply (AVS) conductor to provide greater power for battery charging and higher power needs, and a spare conductor reserved for future use. “LPS” is Low Power Serial, which is a protocol provided within the DSB interface that supplies 3.3 V power to attached devices, rather than 5 V as supplied by the USB protocol. The decision to supply power via the USB or LPS protocol is determined by software present on the DSB Host based upon power requirement information sent to the DSB Host by the attached DSB Device. “DSB Host” refers to a system device that controls the DSB interface, which can be a Patient Worn Hub (PWH) or Smart Display (SD), or a Parameter Transceiver (PT). “DSB Device” refers to a system device that is controlled by a DSB Host through a DSB interface, such as a FED or PWH.

“FED” refers to a front end device, which is a device for collecting patient parameter data. “SD” refers to a Smart Display and is a patient monitor with an integrated display that acts as a DSB Host and runs the algorithms needed for patient parameter processing. An SD can extend the functionality of a Patient Worn Hub (PWH) and provides a larger local display. “HD” refers to a Headless Display and is equivalent to a Smart Display (SD) except that it uses an external display rather than having an integrated display.

In one embodiment, the PWH does not include a display and transmits its data to the monitor which in turn presents said data on the full-screen external display. In one embodiment, to achieve higher acuity monitoring, the PWH is connected to a Smart Display (SD) which provides a larger screen size and additional front end device (FED) ports. In one embodiment, the PWH is connected to the SD via a cable. In another embodiment, the PWH communicates wirelessly with the SD. In another embodiment, the PWH docks to the SD using a docking port. In one embodiment, the SD is a transport type smart display. In another embodiment, the SD is a portable type smart display. In another embodiment, the SD is a fixed type smart display, such as a bedside monitor. The connection to the SD allows the PWH to connect to a hospital network at large so the PWH can publish and archive patient data as well as interact with other devices on the network in real time. Most maintenance (such as software updating) and configuration operations are initiated through the SD user interface using this connection. In another embodiment, the PWH itself contains an internal 802.11 wireless network card so that it can connect to a hospital network.

In one embodiment, the PWH includes an integrated display. In one embodiment, the integrated display is a color touchscreen display.

In one embodiment, the PWH contains an internal NIBP measurement device. In one embodiment, up to five external Front End Devices (FEDs) can be connected to the PWH, allowing the PWH to act both as a system host and as a system device. In one embodiment, caregivers can decrease or increase the number of parameters being monitored as a patient's acuity changes. This is accomplished simply by removing or plugging in more FEDs, creating a “plug and play” environment. In one embodiment, the PWH contains its own internal battery for up to 6 hours run-time and is designed to function as a standalone monitoring device. In one embodiment, the PWH can be connected to a monitor or external display to expand its monitoring and display capabilities.

In one embodiment, the PWH weighs less than 1.2 lbs, in accordance with the current weight standard for handheld devices. The dimensions of the PWH are maintained to ensure easy portability. In one embodiment, the PWH measures less than 5 inches wide by 3.5 inches high by 1.5 inches deep. The PWH is designed to fit comfortably in the hand representative of the largest stature user class and the smallest stature user class, and, in one embodiment, includes a securing band that wraps around the wrist of the user.

In one embodiment, the PWH is designed such that the controls which have the potential to interrupt or prevent proper function are not susceptible to accidental operation. For example, the power on/off interface is designed in such a way to prevent accidental power down while in use. In one embodiment, the power on/off switch is placed within a recess on the surface of the PWH. In one embodiment, the power on/off switch requires a certain amount of force to actuate. In one embodiment, once the power on/off switch is depressed while the unit is in use, the PWH displays a confirmation screen requiring user acceptance before the unit is powered down.

The PWH is designed as a robust device and is capable of operating in hot, cold, dry, and humid environments. In one embodiment, the PWH is capable of surviving a wash cycle in a hospital laundry. In one embodiment, the PWH is capable of being submerged in a liquid tank (e.g., a toilet) for a maximum period of one hour while still retaining functionality. In one embodiment, the PWH can be mounted directly onto installation mounts on the patient monitoring system. In one embodiment, the PWH can be propped up on a flat, horizontal surface, such as a countertop.

In one embodiment, the present specification includes a docking unit for the PWH. In one embodiment, the docking unit provides power to and charges the PWH while the PWH is placed in the docking unit. In one embodiment, the docking unit provides power to and charges the PWH via a cable connected between the docking unit and the PWH. In one embodiment, the docking unit includes its own external power supply.

The PWH's small size and portability allow it to remain with the patient in almost all situations, making it the best choice for storage of the patient data. In one embodiment, the PWH is responsible for storing data collected via directly connected FED's. In another embodiment, the PWH is responsible for storing data collected via directly connected FED's and all data collected from any other FED associated with that patient as well. In one embodiment, the PWH is capable of non-volatile storage of 24 hours of continuous patient data, including waveforms, as well as additional storage for trend data ranging from 24 to 96 hours. In another embodiment, the PWH can connect to a central monitoring system to sync stored patient date, including backfilling any gaps in the patient record.

In one embodiment, a real-time clock (RTC) is integrated into the PWH design to support time stamping of logging and communications. This time-keeping function of the PWH maintains its ability to keep time even when the system battery is not operational. In one embodiment, the PWH is capable of exporting filtered & time synchronized historical data, which includes time-stamped printed tabular trends.

In one embodiment, the PWH supports a plurality of parameter measurements using at least one Front End Device (FED) that is connected into an available FED port located on the PWH. In one embodiment, the PWH includes five FED ports and can therefore support up to five FED's, either proprietary to the applicant or third party. The measurable parameters include, but are not limited to: ECG/respiration (3-lead, 6-lead, and 10-lead); pulse oximetry (SpO₂); temperature (oral, tympanic, and continuous); invasive blood pressure (single channel and 4-channel); cardiac output (thermodilution, continuous, and non-invasive); capnography (mainstream, sidestream, and fraction of inspired oxygen (FiO₂)); mixed venous oxygen saturation (SvO₂) and central venous oxygen saturation (SvcO₂); multi-gas analysis (including anesthetic agent nitrous oxide (N₂O) and O₂); bispectral index (BISx); neuromuscular transmission (NMT); transcutaneous oxygen (TcpO₂) & transcutaneous carbon dioxide (TcpCO₂); spirometry; blood glucose; pulmonary mechanics; electroencephalograph (EEG) and resultant entropy characteristics; auditory evoked potential (AEP) hemodialysis/hemofiltration; and, 16-lead ECG.

In one embodiment, the PWH of the present invention can be connected to third party devices via the use of a ‘living hinge’ connection device, which shall be referred to as a living hinge, hinge connector assembly, or, more generally, as a connection device. The hinge connector assembly comprises device-specific transition connectors. In one embodiment, a third party parameter sensor connection is enabled through the use of a hinge connector assembly, which translates the output of the third party device to the protocol utilized by the PWH or monitor. The hinge connector assembly has a first form when in an unconnected state and then adopts a new form when it is connected to or integrated with other components. This is accomplished by means of a flexible hinge. In one embodiment, the hinge connector assembly is manufactured using a conventional injection molding process.

Once integrated with a cable that connects to a PWH or monitor, the hinge connector assembly becomes a device interface cable. The device interface cable has a DSB connector at one end and a cable connector at the other end to interface with the host and the third party device respectively. In one embodiment, the device interface cable includes a mechanical enclosure that includes printed circuit boards, required cabling with the DSB connector, and the connector to connect with the third party device. The printed circuit boards translate the data provided by the connector of the third party device to the DSB protocol. The mechanical enclosure serves to hold and guide the DSB cable that connects to the PWH or monitor. In one embodiment, the hinge connector assembly includes a groove along the outer circumference of the connector end. The groove holds a gasket which acts to prevent liquid ingress. This simplifies the assembly method by eliminating a secondary operation for assembly of the gasket.

In one embodiment, the hinge connector assembly is designed for single use. Once integrated with the third party device, the hinge connector assembly cannot be disassembled for further use. The PWH can be connected to critical care ventilators, anesthesia delivery systems, hemodynamic devices, infusion pumps, and other devices via the use of the hinge connector assembly.

In one embodiment, the PWH can be connected to a plurality of monitoring peripherals to enhance device functionality, including but not limited to, a mouse, a keyboard (in supported languages), a keyboard with integrated navigation (touchpad or trackball), a barcode scanner, remote control devices, biometric devices, bedside printing devices, and a network laser printer.

In one embodiment, the electrical architecture of the PWH is based upon a single board processor. In addition to the processor, the PWH electrical design also includes a companion chip. The companion chip integrates a number of common system functions into a single chip to allow for compact designs.

In one embodiment, the present invention is directed toward a Patient Worn Hublet device for monitoring patient parameters. The Hublet is a lighter version of the PWH and contains fewer features. In one embodiment, the Hublet includes dedicated hardware and parameter processing for SpO₂ and ECG/Respiration, as well as for NIBP, as these parameters are typically most desired in a monitoring context.

In one embodiment, the Hublet is designed based upon an absolute minimum set of features and is named a Level 1 Hublet. The Level 1 Hublet includes the following features: internal NIBP; internal SpO2; internal ECG/respiration; and minimal FED based parameter support. The Level 1 Hublet does not include any of the following features: integrated display (interfaces via DSB to monitor display); alarm annunciation; stand-alone monitor functionality; or network connectivity.

In another embodiment, the Hublet is designed on a limited features concept and is named a Level 2 Hublet. The Level 2 Hublet includes, but is not limited to, the following features: integrated display with touchscreen; internal NIBP; internal SpO₂; internal ECG/respiration; moderate FED-based parameter support; alarm annunciation; some stand-alone monitor functionality; and limited data storage capability.

The present application is directed toward multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present application is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

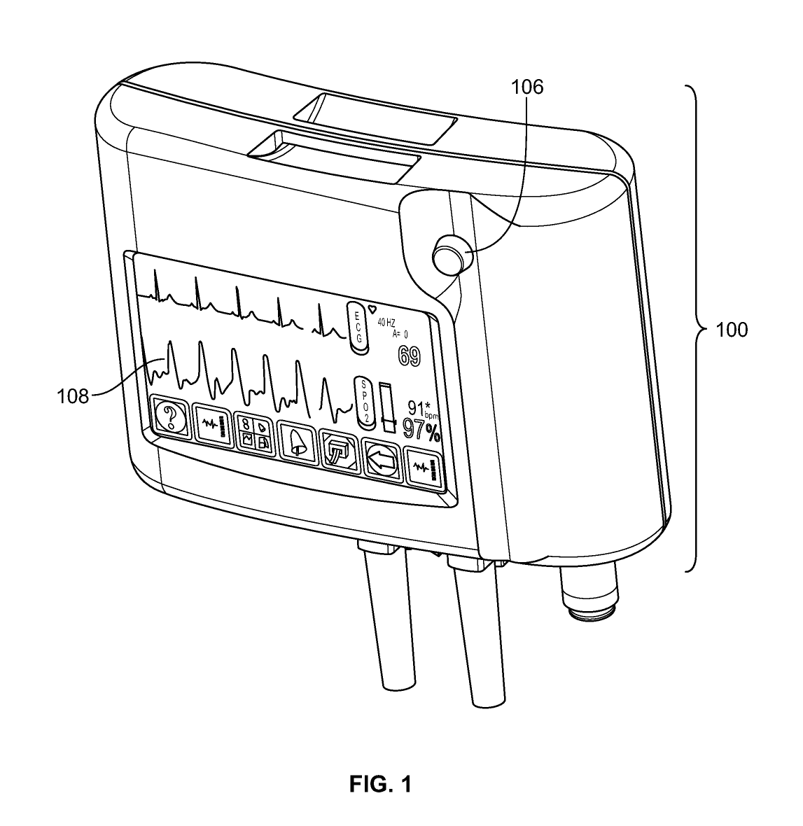

FIG. 1 is an oblique front view illustration of one embodiment of the Patient Worn Hub (PWH) 100. The on/off switch 106 is positioned in a recess on the surface of the PWH 100 so that inadvertent actuation of said switch becomes less likely. This feature helps prevent accidental shut off during use. In this embodiment, the PWH 100 includes an integrated touchscreen display 108. The display 108 features waveform and numerical data and a multitude of touchscreen commands. The on/off switch and touchscreen can be sized and located in different configurations in various embodiments.

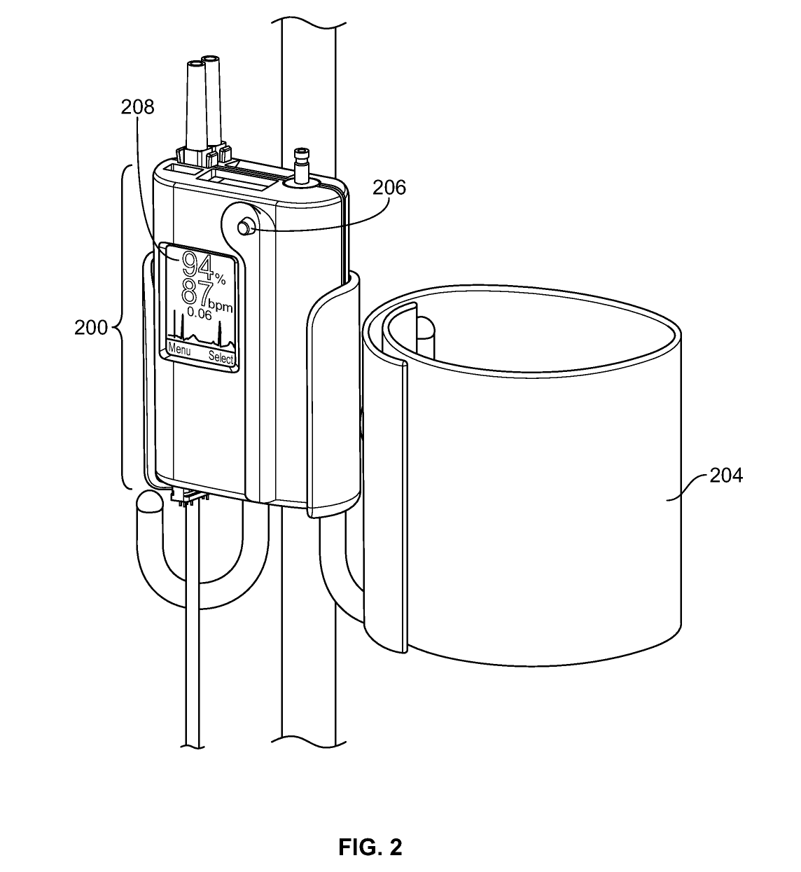

FIG. 2 is an oblique front view illustration of another embodiment of the Patient Worn Hub (PWH) 200 including an optional securing band 204. The securing band 204 is adjustable depending on the arm size of the patient and is used to secure the PWH 200 to the patient during transport and ambulation. This embodiment also includes a recessed on/off switch 206 and an integrated touchscreen display 208.

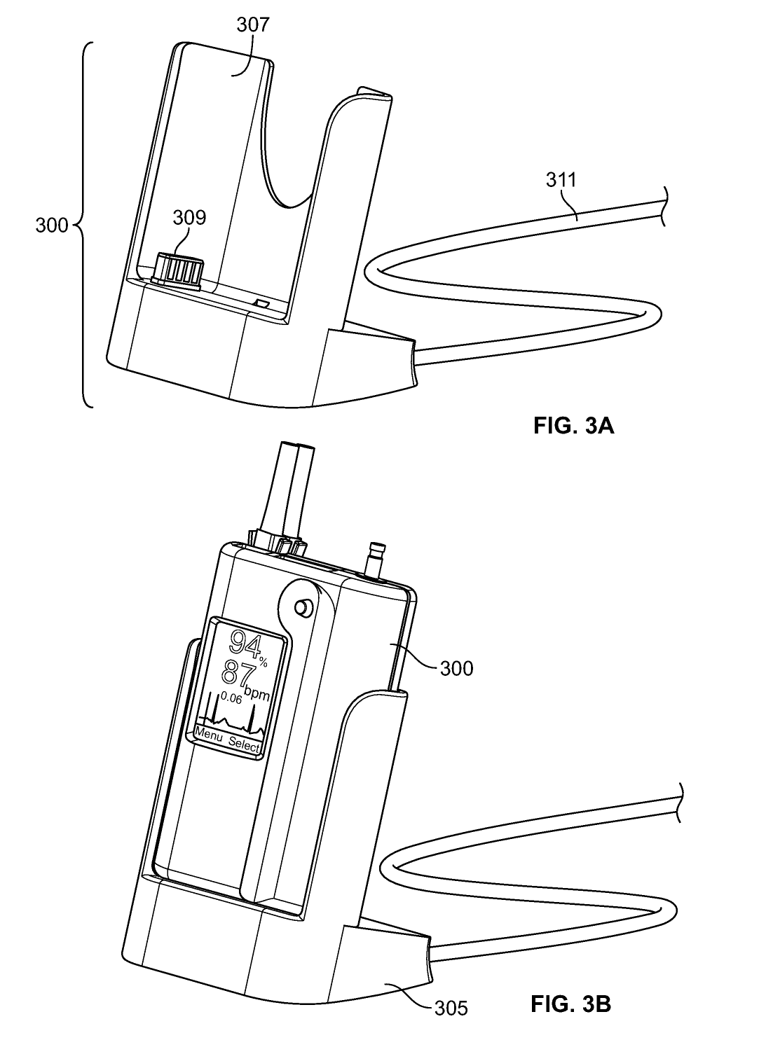

FIG. 3A is an oblique front view illustration of one embodiment of the Patient Worn Hub docking unit 305 of the present invention. The docking unit 305 contains a cradle 307 for holding the PWH and a connector 309 for powering and charging the PWH. The docking unit 305 receives its power directly via an attached power cord 311.

FIG. 3B is an oblique front view illustration of one embodiment of the Patient Worn Hub docking unit 305 depicting the PWH 300 placed into said docking unit 305. The PWH 300 fits snugly into the docking unit 305 and receives power and charging while placed into the docking unit 305.

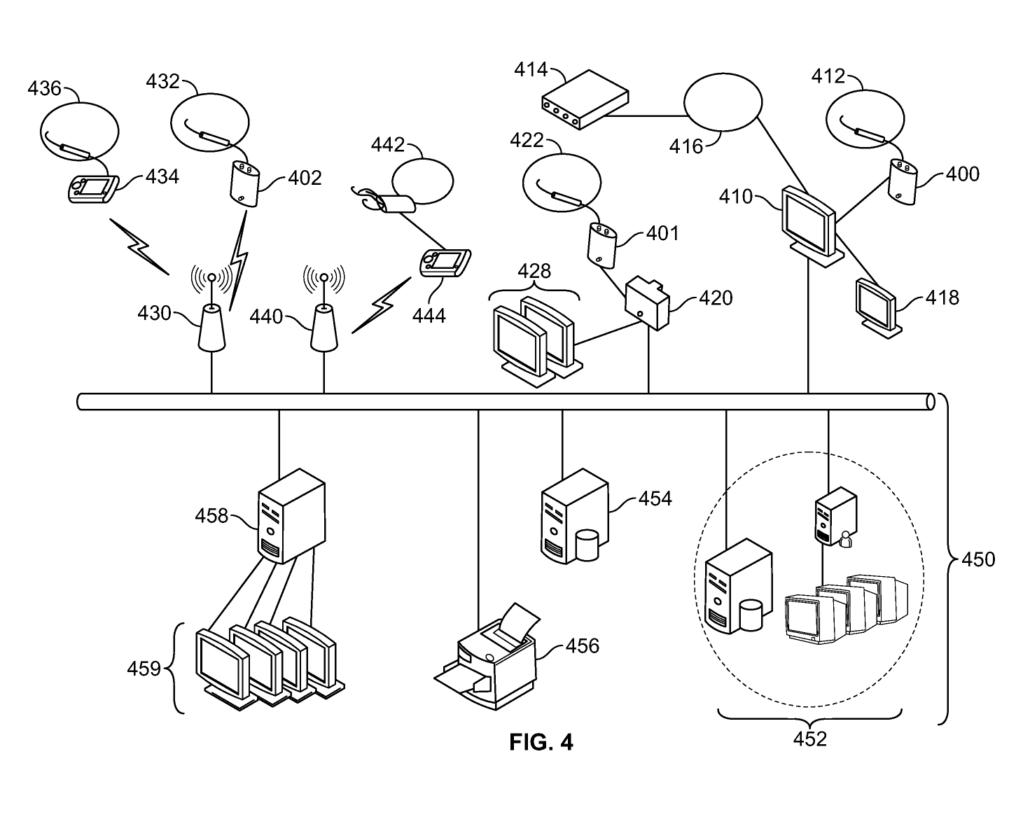

FIG. 4 is an illustration of a multitude of possible embodiments of the Patient Worn Hub 400, 401, 402 connected to a hospital network 450. The hospital network 450 includes legacy systems 452, a central data store (DB) 454, a printer 456, and a central station (CS) 458 with a multitude of displays 459. In one embodiment, the PWH 400 is connected to a Smart Display (SD) 410 which is in turn connected to the hospital network 450. An FED 412 is connected to the PWH 400, via a hinge connector assembly as described above, to measure patient parameters. In addition, a third party device 414 is connected to the SD 410 via a device interface (DI) 416, which also includes a hinge connector assembly as described above. An additional display 418 is also connected to the SD 410.

In another embodiment, the PWH 401 is connected to a Headless Display (HD) 420 which is in turn connected to the hospital network 450. An FED 422 is connected to the PWH 401 to measure patient parameters. Additional displays 428 are connected to the HD 420.

In another embodiment, the PWH 402 communicates wirelessly with a wireless access point 430 which is in turn connected to the hospital network 450. An FED 432 is connected to the PWH 402 to measure patient parameters. A Parameter Transceiver (PT) 434 also communicates wirelessly with the IPAP 430. An additional FED 436 is connected to the PT 434 to measure patient parameters.

In another embodiment, a Parameter Transceiver (PT) 444 communicates wirelessly with a wireless medical device access point (MDAP) 440 which is in turn connected to the hospital network 450. An FED 442 is connected to the PT 444 to measure patient parameters.

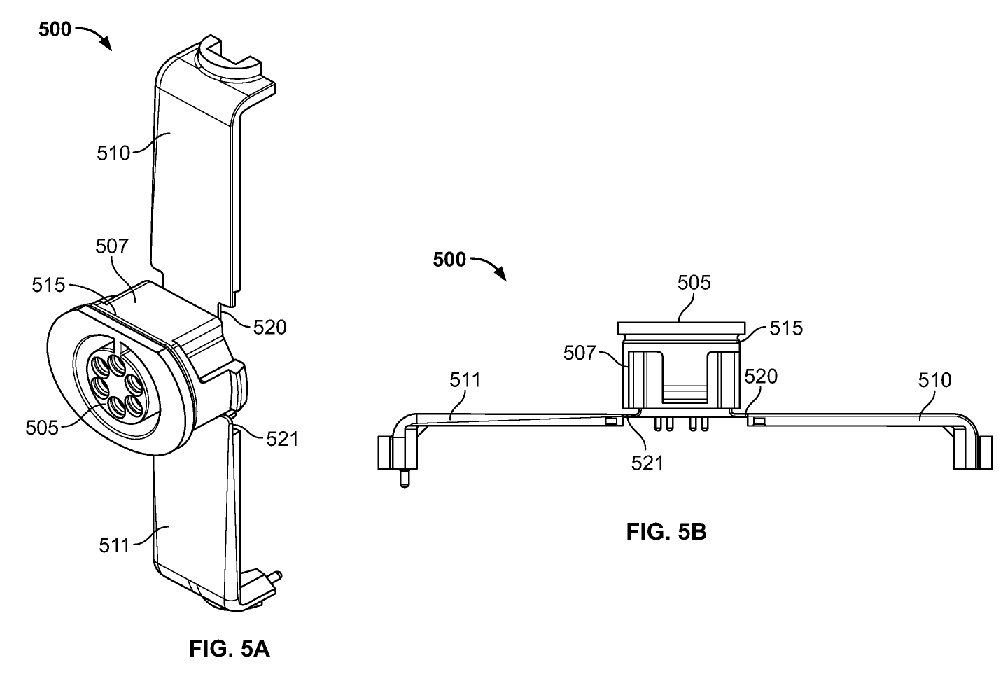

FIGS. 5A and 5B depict oblique front view and side view illustrations, respectively, of one embodiment of the hinge connector assembly 500 in the open position prior to assembly. In one embodiment, the hinge connector assembly 500 includes an elevated plug member 507, to which are attached an upper wing member 510 and a lower wing member 511. In one embodiment, the elevated plug member 507 is formed in the shape of an oval. In one embodiment, a connector 505 is set into the face of the elevated plug member 507 for connection of a third party device. In one embodiment, the connector 505 is formed in a circular shape with six inserts. In various other embodiments, the elevated plug member and connector can take any shape that facilitates connection to a desired third party device. In one embodiment, a gasket 515 is included around the end of the elevated plug member 507 for protection against liquid ingress.

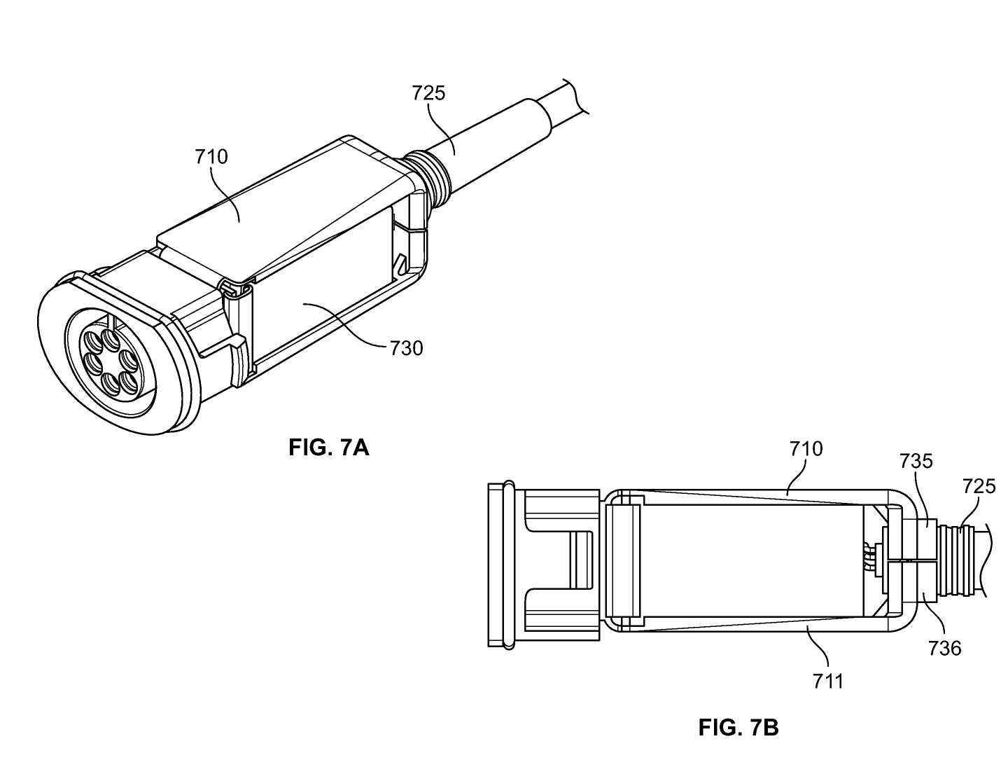

The upper wing member 510 is movable downward and away from the front of the plug member 507 about an upper hinge 520 in the range of 90 degrees from its depicted fully extended and open configuration to a closed, operable configuration as seen in FIGS. 7A and 7B. The lower wing member 511 is movable upward and away from the front of the plug member 507 about a lower hinge 521 in the range of 90 degrees from its depicted fully extended and open configuration to a closed, operable configuration as seen in FIGS. 7A and 7B.

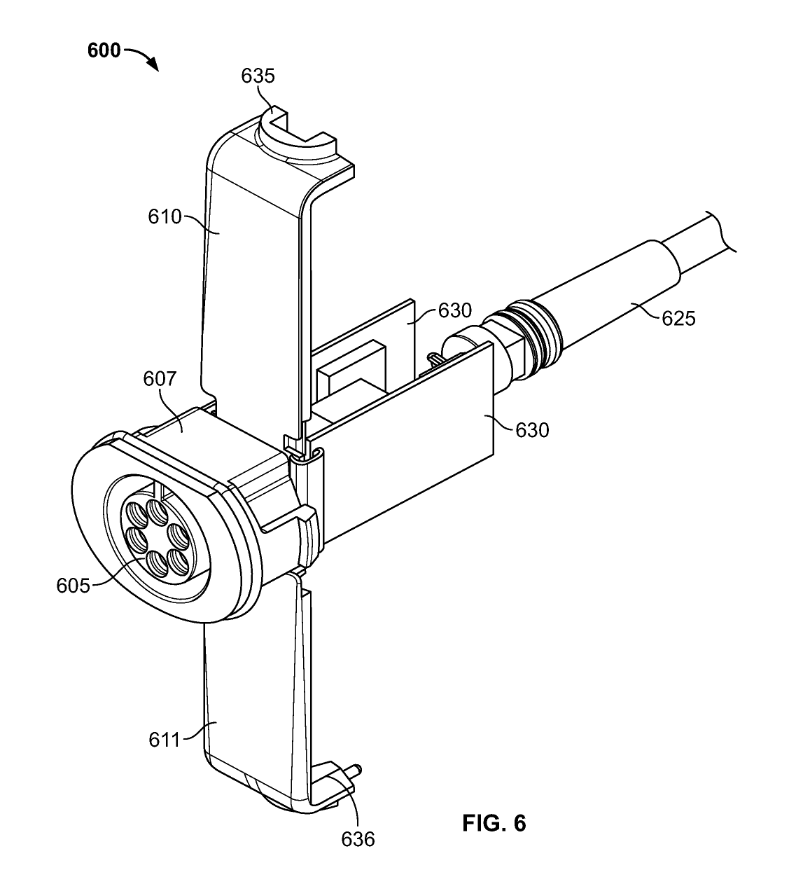

FIG. 6 is an oblique front view illustration of one embodiment of the hinge connector assembly 600 in the open position with a DSB device cable 625 in place. Also depicted are the printed circuit boards (PCB) 630 required to translate the output of the third party device to the protocol embedded within the PWH. In one embodiment, the PCBs 630 are oriented perpendicular to the plane of the wings 610, 611 and attach to the elevated plug member 607 on the side opposite the connector 605. The DSB cable 625 interfaces with a connector within the enclosure formed by the PCBs 630 and the wings 610, 611. The wings 610, 611 fold in 90 degrees toward the PCB's 630 and the DSB device cable 625 to complete assemblage of the hinge connector assembly. At the end of the upper wing—610 is a circular shaped cutout 635 with holes on either side that interfaces with a similarly shaped cutout 636 with pins on either side located at the end of the lower wing 611 to form a DSB device cable 625 holding feature when fully assembled.

FIGS. 7A and 7B are oblique front view and side view illustrations, respectively, of one embodiment of the hinge connector assembly fully assembled with a DSB device cable 725 attached. In the fully assembled position, the folded wings 710, 711 form an enclosure, like a chassis, around the PCB's 730 and DSB device cable 725. This enclosure acts to guide and securely fix the PCB's 730 and the DSB device cable 725 in place. The ends of the wings 710, 711 include cutouts 735, 736 respectively that interface to hold the DSB device cable 725 in place. In one embodiment, these cutouts 735, 736 include a set of pins and holes on either side which interface when the wings 710, 711 are in the assembled position.

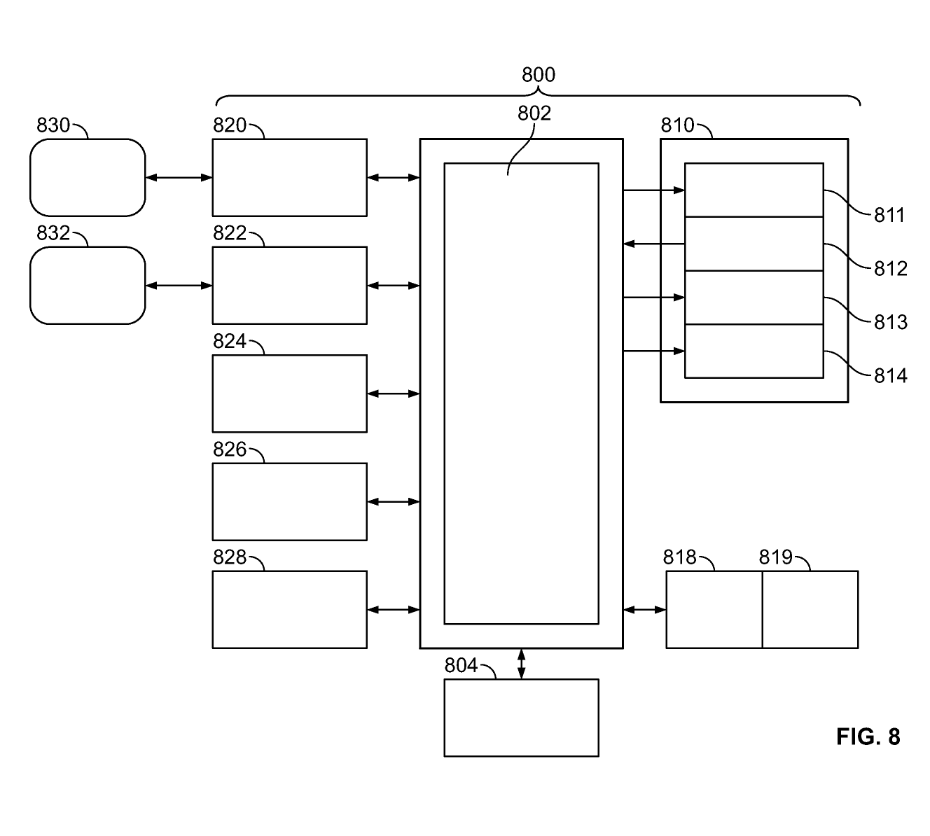

FIG. 8 is a block diagram illustrating the components included in one embodiment of the Patient Worn Hub 800. The PWH 800 comprises at its core a processor 802 which is coupled with a storage module 804, a user interface 810, a DSB device port interface 820, a DSB host port interface 822, a power management module 824, a wireless Ethernet 824, a battery charger/battery gas gauge 828, and a serial port interface 818. The core processor 802 processes, stores, and displays all the data supplied by the associated FEDs. In one embodiment, the processor core 802 is not active more than 50% of the time as averaged over a one second interval in a worst case configuration. The PWH 800 processor 802 manages its power utilization effectively, moving to lower power modes when possible. In one embodiment, the processor 802 operates under the Windows® CE operating system.

In one embodiment, the storage module 804 comprises non-volatile storage, such as Flash™ memory, which is used to store all collected and processed patient data as well as programs (including boot code), back-up programs, and all information related to those programs. The processor 802 is capable of booting from the non-volatile memory. In one embodiment, the storage module 804 also comprises volatile (RAM) storage large enough to accommodate the runtime memory requirements of the processor 802. This includes space for uncompressed executable operating system and application code. In one embodiment, the non-volatile memory storage is capable of storing complete clinical history data including all waveform and numeric physiological data for a minimum of 24 hours as well as additional storage of numeric trend data only for a minimum of 96 hours. Further, the non-volatile memory storage stores duplicate software images, including supporting non-volatile databases, languages, and configuration information.

In one embodiment, the user interface module 810 comprises a display module 811, a touchscreen module 812, an audio module 813, and an alarm LED module 814. The display module 811, in one embodiment, is a full color display which operates primarily in landscape mode and can operate in portrait mode. The core processor 802 interfaces to this display 811 through an internal image processing unit (IPU), while a graphics processing unit (GPU) provides hardware support for image rendering. In one embodiment, the PWH 800 is provided with a backlight. This backlight driver circuit supports adjustable brightness so the display screen 811 may be read easily in any hospital environment.

In one embodiment, user interaction is primarily accomplished using the touchscreen module 812 on the PWH 800. The touchscreen 812 can be locked to prevent accidental interaction by selecting the appropriate function through the touchscreen interface. Once locked, a series of touchscreen selections will be necessary to unlock the screen so it will not be inadvertently unlocked. The touchscreen 812 does not require a special device, such as a stylus, to operate and can be operated by finger contact. In addition to showing the patient information in numeric and waveform formats, the touchscreen 812 includes a multitude of control and status functions. In one embodiment, the control of an attached non-invasive blood pressure measurement module of the PWH 800 is through the touchscreen 812. In one embodiment, the PWH 800 provides status information such as battery condition and charge state through the touchscreen 812 interface enabling the user to determine the amount of time remaining while operating on batteries or the charge state when plugged into an upstream or charging device. The PWH 800 alerts the user when the battery level is too low or when the battery needs to be replaced. In one embodiment, the alert is an audio-visual alarm that is displayed on the screen to give an indication that the battery life is nearing end.

In one embodiment, the audio module 813 comprises an audio output section that is used for alarm annunciation and user feedback (such as a tone to indicate acknowledgement of a user action). In one embodiment, the audio output supports an adjustable volume. The audio module 813 is also capable of emitting distinct audible tones based upon the source of the alarm. It is possible to control the volume and to mute the alarm from the touchscreen user interface 812. It is also possible to adjust alarm volumes and alarm status using a touchscreen interface 812. In one embodiment, high, medium and low priority alarms all have distinct alarm tones associated therewith to yield a clear distinction between alarms.

In various embodiments, the PWH 800 also provides visual indicators during an alarm situation. In addition to showing alarm information on the display module 811, the PWH 800 will provide visual indications on the case so an alarm will be evident in a situation where the PWH 800 is resting with the display module 811 face down. In one embodiment, the alarm LED module 814 comprises an array of LEDs. In one embodiment, the LED's support red, green, and blue colors. In multiple embodiments, various alarm situations can be represented by various combinations of colors. The LED's emit light through a semi-transparent section of the enclosure, such that the sides of the PWH 800 are illuminated when the LED's are active.

In one embodiment, the PWH 800 includes an upstream port 820 and a downstream port 822. The upstream port 820 is a DSB connection to Host devices 830 such as a Smart Display and is not Low Power Serial (LPS) compatible. The downstream port 822 connects to the external devices 832 such as FED's, and is LPS compatible. The upstream ports are designed to conform to the DSB specification and are LPS capable. The DSB device port interface 820 provides an upstream DSB port for connection to Smart Displays or other similar types of monitoring devices, either by cable or by docking the PWH 800 into the Smart Display. This DSB device port 820 is compliant with the USB high speed and the DSB specifications (excluding the LPS portion). In one embodiment, the Smart Display or similar device may reset the PWH 800 without intervention from the PWH processor 802. The PWH 800 also may be charged through the upstream DSB device port 820.

In one embodiment, the DSB host port interface 822 contains multiple downstream DSB ports for connection of external devices 832 such as FED's. The DSB ports allow for connection and disconnection of FED's regardless of the operational state of the PWH 800. Since each DSB port supports USB (full and low speed), any USB device may be plugged in as long as it conforms to the DSB specification and interface control specifications. When an FED 832 is plugged in, the USB subsystem in the PWH core processor 802 initiates contact with the FED 832. The FED 832 will then request power (if necessary), and the PWH 800 will respond by either denying the request or by enabling either the higher current USB or by enabling an Auxiliary Voltage Supply (AVS) line. Once the hardware configuration is established, both sides finish initialization and switch to normal runtime activities. When an FED 832 is disconnected, the corresponding port reverts to a disconnected state and the AVS line is powered down (if powered). The PWH 800 design accommodates the appropriate line filtering and/or isolation to allow plugging and unplugging of DSB devices without affecting other unrelated functions. In one embodiment, the PWH 800 can support external user interface devices (such as a barcode reader) via the downstream ports. In one embodiment, the PWH 800 provides a method of uniquely authenticating users through (but not limited to) fingerprint (biometric) identification, radio frequency ID tags, bar-code readers, or user passwords.

In one embodiment, the USB host port interface 822 supports a full speed/low speed connection to externally connected FED's. In one embodiment, an internal 7 port hub provides the expansion out to the 5 DSB ports. This hub provides power control for each DSB port, limiting the power to 500 mA per port. Over current detection is provided for each port through this internal hub. Additionally, each port has on/off control, which provides the PWH 800 the ability to reset a connected FED. The PWH 800 additionally provides AVS power to each DSB port. The AVS supply is initially off, but can be enabled on a port-by-port basis. The PWH 800 limits the AVS output power per port. Over current detection for the AVS line is provided for each port.

The PWH 800 is self-powered so it may continuously monitor a patient in any normal hospital situation. When connected to the Smart Display, the PWH 800 uses the SD's AVS line to power the PWH functions and uses any extra available AVS power to charge the batteries. In one embodiment, the PWH 800 may be powered from the AVS supply of the upstream port 820, from which it will supply power to all internal PWH functions as well as the external DSB functions. The PWH 800 is also provided an internal battery, from which it can supply power to all internal PWH functions as well as the external DSB functions. The power management module 824 provides the functionality of conserving power, including but not limited to, temporarily decreasing or turning off the backlight for the display and disabling clocks to idle interior devices. The PWH 800, when connected to an SD, or stand-alone charger, may remain in an operational state while charging. In one embodiment, the internal battery may be charged from a fully discharged state to a fully charged state in 1.5 hours or less when the PWH 800 is connected to a suitable external power source. In one embodiment, a battery gauge module 828 enables the PWH 800 to accurately track battery life and charge state.

Unlike a standard laptop computer or a cell phone, which operate infrequently and spend much of their time in an idle state, the PWH 800 will spend much of its time collecting and processing data. In order to maximize the battery life of the PWH 800 or connected SD, the PWH 800 powers down any unused function (as much as is feasible), including the display. In cases where the core processor 802 is in a powered on but extended idle state, the core processor 802 is placed in a standby mode. In this mode, any interaction from an FED 832 or a user interface such as the touchscreen 812 places the PWH 800 back into an operational state.

In one embodiment, the PWH 800 provides a wireless Ethernet radio 826. This enables wireless connection to a main control system via a wireless access point. The PWH 800 provides an internal antenna for this radio. In one embodiment, the PWH 800 additionally provides capability for a wired Ethernet connection.

In one embodiment, a non-invasive blood pressure measurement (NIBP) FED 819 is housed within the PWH enclosure. The NIBP FED 819 is controlled by a separate microcontroller that communicates with the PWH core processor 802 via an internal serial port 818 connection. Control information, data, and maintenance messaging to and from the NIBP 819 processor is all funneled through the serial port 818. The PWH 800 supports adult, pediatric, and neonatal patients. Configuration of the NIBP 819 for functions such as sample interval and patient type (adult, pediatric, etc.) are made through the PWH display 811/touchscreen 812 interface. Although the NIBP 819 control is mostly autonomous with respect to the PWH processor 802, the PWH processor 802 is used to provide the “safety processor” functions for the NIBP 819 sub-system. The PWH processor 802 monitors the secondary pressure port and can override and deflate the external NIBP cuff if it detects an error condition. The processor 802 assures that communication with the NIBP 819 is active, and if not, an alarm is signaled and the NIBP 819 pump and valve control are overridden. This insures that the NIBP 819 does not fall into a harmful situation in the event of a problem with any part of the NIBP 819 system.

In one embodiment, the PWH 800 may reset the internal modules such as DSB hubs 820/822 or the NIBP 819 processor as well as external devices 832 such as any connected FED. Further, the PWH 800 may be reset by a command from the display interface 811, from the ON/OFF button, or from a Smart Display device. In one embodiment, the PWH 800 is provided with latch circuitry, which may be accessed by the core processor 802 to determine if the reset was initiated via a reset line or from a power condition. The core processor 802, upon initialization, will change the state of the latch from its default setting. Upon recovery from a reset, if the core processor 802 notes that this latch is in its default state, then the latch itself must have reset due to a power condition. If it is in a non-default state, then the reset must have been a commanded reset, such as a “soft” or “hard” reset. In one embodiment, the reset mechanism for the Smart Display operates even in cases where the core processor 802 is non-functional, by detecting the case where the upstream AVS line is powered and the BUS line goes low. This low period must extend long enough to avoid false triggering. In order to reset a connected FED 832, the PWH 800 simply drops the BUS line out to the FED 832 for a period of time, and then re-enables this line. In various embodiments, reset information may be retrieved for diagnostic purposes. The reset information may be accessible via the user interface 810.

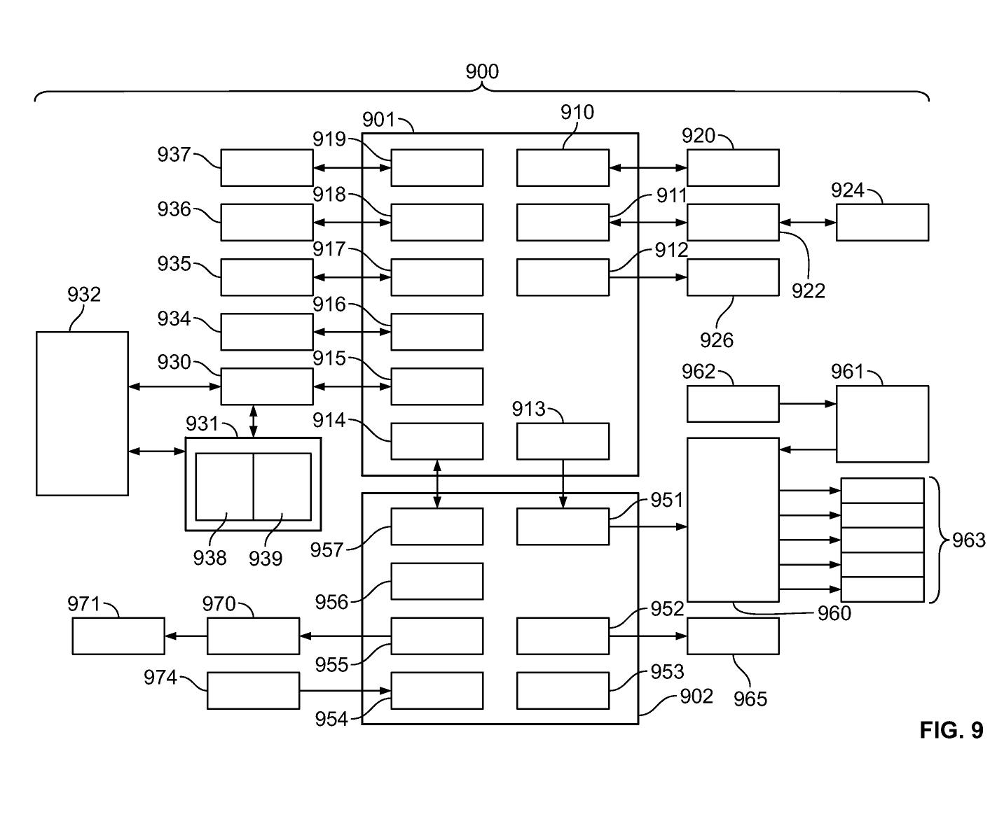

FIG. 9 is a block diagram illustrating one embodiment of the design of the electrical architecture of the PWH 900. This embodiment includes the CPU 901 and the companion chip 902. In one embodiment, the CPU 901 includes the following components: a memory interface (I/F) 910 which communicates with memory 920; a DSB Client 911 which communicates with a DSB hub controller 922 which in turn communicates with a DSB Connector (to SD) 924; a general purpose interface 912 which communicates with switches and LED's 926; a core voltage/frequency controller 913 which controls the power management component 951 of the companion chip 902; a serial peripheral interface (SPI) 914 which communicates with the CPU interface 957 of the companion chip 902; a DSB Host 915 which communicates with a DSB Hub 930 which in turn communicates with both an external parameter device power control component 931 and DSB connections to external parameter devices 932; another SPI 916 which communicates with a wireless local area network (WLAN) 934; a universal asynchronous receiver/transmitter (UART1) 917 which communicates with a non-invasive blood pressure (NIBP) measuring device 935; an image processing unit (IPU) 918 in communication with a display 936; and a Flash controller 919 in communication with Flash memory 937. The external parameter device power control component 931 includes +5 V hi/Lo power controllers 938 and one +18 V hi/lo power controller 939, and regulates power to the DSB connections for external parameter devices 932.

In one embodiment, the companion chip 902 includes the following components: a power management component 951 controlled by the core voltage/frequency controller 913 of the micro-controller central processing unit (CPU) 901; a backlight power supply 952 which provides power to the display backlights 965; a real-time clock (RTC) 953; a touchscreen controller 954 which communicates with the touchscreen 974; an audio out 955 which sends signals to the amp 970 which in turn sends signals to the speaker 971; a codec 956; and, a CPU interface 957 which communicates with the serial peripheral interface (SPI) 914 of the micro-controller CPU 901. The power management component 951 controls the power supply 960 which determines a range of power output 963 (1.2-1.65, 1.8, 3.3, 5, and 18 V). A battery charger 962 charges a lithium ion (Li-Ion) battery 961 which in turn supplies power to the power supply 960.

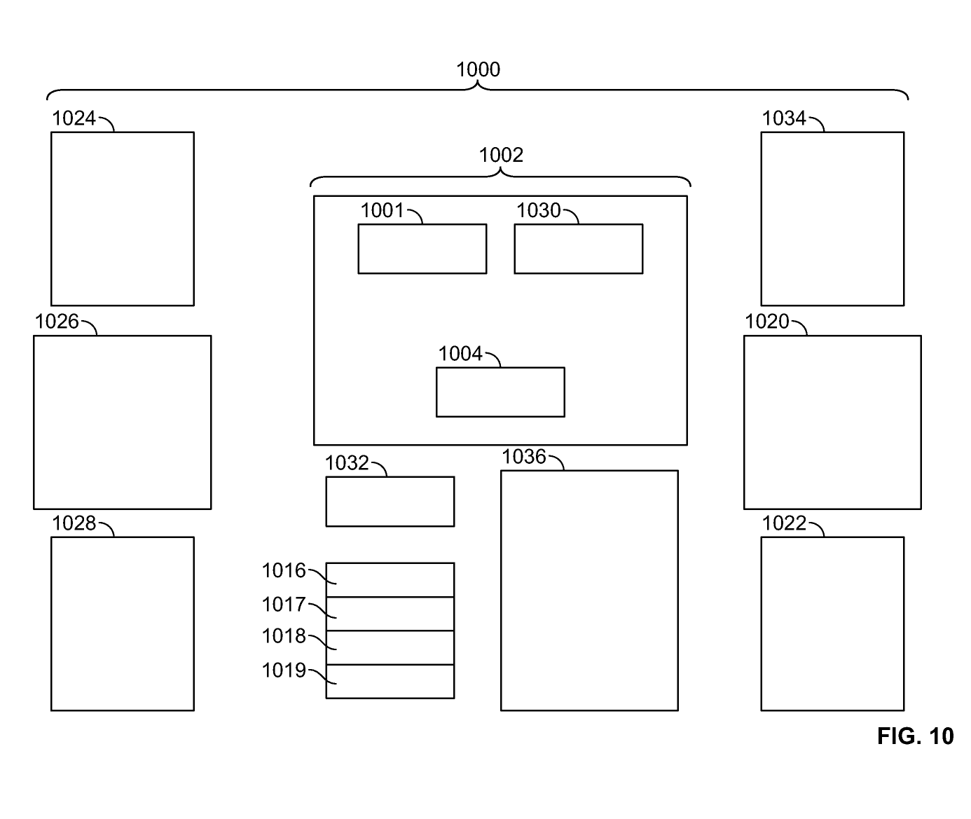

FIG. 10 is a block diagram illustrating one embodiment of the processor 1000 included in the electrical architecture of the PWH. The processor 1000 is based upon a core 1001 architecture, which supports the Windows® CE operating system. In one embodiment, the CPU core 1002 is augmented with a co-processor 1004.

In one embodiment, the processor 1000 has memory interfaces for synchronous dynamic random access memory (SDRAM)/double data rate random access memory (DDR RAM) 1016, pseudostatic random access memory (PSRAM) 1018, and SmartMedia 1019, as well as a built in NAND flash controller 1017. In one embodiment, the PWH is provided with two separate Flash memory devices: a NAND Flash and a NOR Flash. In one embodiment, the 1 GB NAND flash contains all information associated with the patient including the trend and historical data. No other external devices are needed for the NAND Flash since the micro-controller contains a NAND flash controller. The micro-controller 1000 cannot boot from the NAND Flash due to its page access interface, so a NOR Flash is required. Since this device is necessary, it is preferable to store the code images in the NOR Flash. The Flash memory contains the boot software image, main software image, and backup software image for the PWH. In one embodiment, the NOR Flash is available with a 1.8V interface, so no external devices are needed to interface this to the CPU. In one embodiment, the micro-controller processor 1000 also provides a Dual Data Rate (DDR) interface. This interface provides data on both edges of a clock cycle. Since many of the Mobile DDR devices are available with a 1.8V interface, no additional hardware is necessary to connect the DDR to the processor.

In one embodiment, the display support included in the processor 1000 consists of a multimedia and human interface 1020 and an Image Processing Unit (IPU) 1022. In one embodiment, the multimedia and human interface 1020 comprises an internal Graphics Processing Unit (GPU) which provides hardware acceleration for many graphics functions, an MPEG-4 Encoder, and a Keypad interface. In one embodiment, the IPU 1022 handles image manipulation (inversion, rotation, blending, scaling, etc.), Pre- and Post-Processing, and a Camera interface, as well as image output to the display via a Display/TV Controller.

In one embodiment, the processor 1000 provides internal connectivity interfaces 1024 for connecting internal devices as well as external connectivity interfaces 1026 for connecting external devices. In one embodiment, the internal connectivity interfaces 1024 include three CSPI interfaces, three I²C interfaces, two SSI/I²S interfaces, and one Audio Mux interface. In one embodiment, the external connectivity interfaces 1026 include five UART interfaces, one USB OTG HS interface, two USB Host interfaces, one 1-Wire interface, and one Fast IrDA interface. Higher throughput devices can be connected via expansion interfaces 1028 through one PCMCIA/CF port, two MMC/SD ports, two Memory Stick Pro ports, one SIM port, and one ATA port. Two of the three USB ports are host-only ports supporting USB high speed connections, while one of the high speed ports can be configured as a device (for connection to upstream USB devices). Supporting the interfaces 1024, 1026 are an array of DMA controllers and a 5 input, 6 output crossbar connection smart speed switch 1030 which allows simultaneous access to multiple devices.

In one embodiment, to support security, the processor 1000 has built in security hardware support 1032 for high assurance boot (HAB), runtime integrity checking (RTIC), and tamper detection. The HAB authenticates the boot code, preventing booting from unauthorized code. The RTIC checks sections of memory during execution. If the contents fail to match the original hash signature, the security monitor is triggered. The tamper protection allows the change of an external IO pin (selectable) on the processor to trigger the security monitor.

In one embodiment, the processor 1000 supports standard system control functions 1034 including joint tag action group (JTAG), electronic token meter (ETM), bootstrap, system reset, and phase-locked loop (PLL) and power management. In one embodiment, the processor 1000 supports standard system input/output (I/O) functions 1036 including direct memory access (DMA), three timers, pulse-width modulation (PWM), watchdog (WD) Timer, real time clock (RTC), general purpose input/output (GPIO), and random access memory (RAM)/read-only memory (ROM).

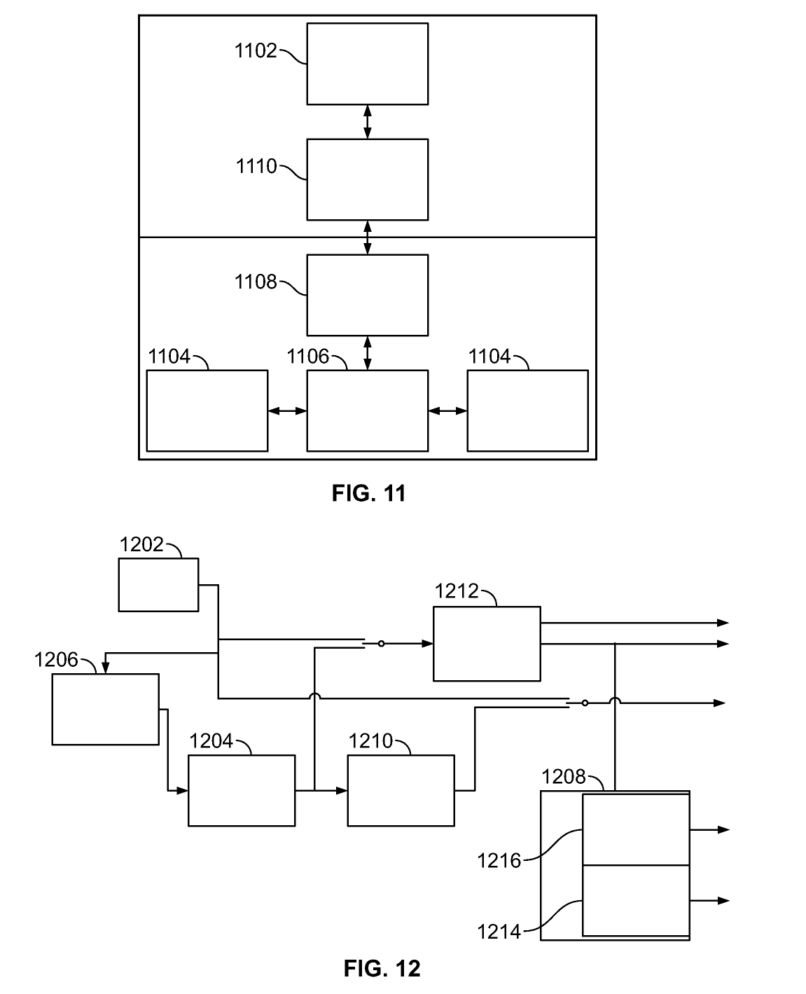

FIG. 11 is a block diagram illustrating one embodiment of an upstream USB port of the PWH. The upstream USB port 1102 of the PWH provides a USB compliant high speed connection to upstream devices 1104. The port on the micro-controller CPU is connected via a multiplexer (MUX) 1106 either to a standard DSB connection or to a set of contacts that provide connection when the PWH is docked in a Smart Display (SD) type of device. The port MUX 1106 switches to the connected port when it senses that port's VBUS line. If both the standard DSB connection and the SD contacts are active, the standard DSB port is selected. The VBUS lines of each port are connected through diodes to prevent feedback of the VBUS supply in the case of a dual connection. When the PWH connects to an upstream device, it must enumerate as a PWH before the connected device enables its AVS power. This presents a problem for a PWH that is too low on battery power to operate. For this reason, a bus powered USB High Speed hub 1108 is inserted in between the MUX 1106 and the USB port 1102 on the micro-controller so that it may enumerate even when the rest of the PWH is powered off. An electrically-erasable programmable read-only memory (EEPROM) connected to the Hub provides the vendor identification (VID)/product information (PID) for proper enumeration. In order to connect the micro-controller port 1102 to the High Speed Hub 1108, a USB High Speed transceiver 1110 is needed. This transceiver 1110 changes the data from a serial stream to a byte wide stream that operates at a lower rate. The transceiver 1110 also combines the CPUs transmit and receive paths into a single differential output. The USB port 1102 and transceiver 1110 are internally powered and the USB Hub 1108, MUX 1106, and upstream devices 1104 are all input VBUS powered.

FIG. 12 is a block diagram illustrating one embodiment of the power section of the PWH. The power section of the PWH requires a number of voltage levels for the external devices while the core processor itself requires the power be segmented into banks. The main power source for the PWH emanates from the upstream DSB port's AVS line 1202, or from the internal PWH battery pack 1204. In one embodiment, the battery pack 1204 comprises a 2-cell lithium ion (Li-Ion) battery pack. The batteries are connected in series, providing a maximum voltage of 8.4 V and a minimum working voltage of 6 V. The battery pack 1204 contains a safety circuit as well as a highly accurate gas gauge. This gas gauge communicates with a battery charger 1206 through a system management bus (SMBUS) connection. The gas gauge contains information about the battery pack 1204 as well as vendor specific information, which allows for differentiation between valid and counterfeit battery packs.

In one embodiment, the battery charger 1206 is an SMBUS compliant “smart charger”. This charger 1206 is automatic from a user point of view. The core processor detects when the batteries need to be charged and enables the charger if running on external power. Charge profiles are programmable via the SMBUS interface. In order to keep the PWH operational while charging, the battery charger 1206 gets only the remaining current above what is necessary for operation. In various embodiments, the PWH has the ability to reduce power consumption to the lowest possible levels in order to minimize battery size and maximize run-time. Based upon processor activity, the PWH core processor automatically manages the core power in conjunction with the micro-controllers companion chip 1208. When practical, the CPU may turn off external devices (such as the Flash memory, Display, USB Hubs, etc.) when not in use.

The core processor possesses the ability to power down each voltage regulator, except those regulators on which it will depend for operation. The PWH also uses the AVS input 1202 to drive the AVS outputs to the FEDs. If the input AVS 1202 is not available, then a boost converter 1210 is used to boost the battery supply voltage up to the level required for the AVS outputs. To do this, the battery pack supply is fed into a boost-style switching regulator to boost the voltage. The cutover to this regulator is automatic, based upon the presence of the AVS input. Additionally, when this input is available, the voltage regulator is disabled to save power. Due to the wide input range a switching type regulator 1212, is provided. The output primarily supplies the VBUS lines for the downstream USB ports as well as any components that require 5 V supplies, such as the audio amp. The 3.3 V regulator supplies all 3.3 V components as well as providing the input for the micro-controller companion chip 1208. The companion chip 1208 provides a plurality of integrated linear and switching style regulators. Regulator 1214 provides the 1.8 V required for the PWH as well as supplies the core processor's I/O pins. Regulator 1216 is a dynamically controlled regulator, which provides the 1.2 to 1.6 V output which is used to power the core processor.

The above examples are merely illustrative of the many applications of the system of the present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

1. A portable, self-contained device used to monitor patient parameters, comprising an internal battery, at least one connection for a parameter measuring instrument, and at least one connection assembly for connection of a third party measuring instrument, said connection assembly comprising printed circuit boards, a plug, and an enclosure, wherein said device:
a. provides continuous, uninterrupted monitoring of at least one patient parameter;
b. is capable of communicating with a bedside monitor and a patient monitoring network;
c. is worn by and remains with an assigned patient for the duration of care; and,
d. is designed in a compact and lightweight manner to enhance portability.

2. The portable, self-contained device used to monitor patient parameters of claim 1, wherein said device comprises a connector for docking of said device within a docking station, wherein said docking station provides operating and battery charging power to said device, and wherein said docking station receives power from an external power supply.

3. The portable, self-contained device used to monitor patient parameters of claim 1, wherein said patient parameters comprise electrocardiograph (ECG)/respiration, pulse oximetry (SpO₂), temperature, invasive blood pressure (IBP), non-invasive blood pressure (NIBP), cardiac output, capnography, mixed venous oxygen saturation (SvO₂) and central venous oxygen saturation (SvcO₂), multi-gas analysis, bispectral index (BISx), neuromuscular transmission (NMT), transcutaneous oxygen TcpO₂ & transcutaneous carbon dioxide (TcpCO₂), spirometry, blood glucose, pulmonary mechanics, electroencephalograph (EEG) and resultant entropy characteristics, auditory evoked potential (AEP), hemodialysis/hemofiltration, and 16-Lead ECG.

4. The portable, self-contained device used to monitor patient parameters of claim 1, further comprising an integrated touchscreen display.

5. The portable, self-contained device used to monitor patient parameters of claim 1, further comprising alarm annunciation capabilities.

6. The portable, self-contained device used to monitor patient parameters of claim 1, further comprising an integrated NIBP measuring instrument.

7. The portable, self-contained device used to monitor patient parameters of claim 1, further comprising internal memory capable of storing measured patient parameter data.

8. The portable, self-contained device used to monitor patient parameters of claim 5, wherein said internal memory is capable of storing a minimum of 24 hours of continuous patient data and a minimum of 96 hours of trend data.

9. The portable, self-contained device used to monitor patient parameters of claim 1, further comprising an internal real-time clock (RTC).

10. The portable, self-contained device used to monitor patient parameters of claim 1, wherein said device communicates through a wired connection with said bedside monitor via detachable cables.

11. The portable, self-contained device used to monitor patient parameters of claim 1, wherein said device further comprises an internal wireless network card and said device is capable of communicating wirelessly with said bedside monitor, a parameter transceiver, and a hospital network.

12. The portable, self-contained device used to monitor patient parameters of claim 11, wherein measured data is transmitted from said device to said bedside monitor and then presented in numerical and graphical form on an external display.

13. The portable, self-contained device used to monitor patient parameters of claim 1, wherein said parameter measuring instrument is connected to said device via a Dual Serial Bus (DSB) connection.

14. The portable, self-contained device used to monitor patient parameters of claim 1, further comprising a securing band or similar structure for securing the device to the patient during transport and ambulation.

15. A portable, self-contained device used to monitor patient parameters, wherein: said device comprises an internal battery, an integrated NIBP measuring instrument, an integrated SpO₂ measuring instrument, an integrated ECG/Respiration instrument, at least one connection for a parameter measuring instrument, and at least one connection assembly for connection of a third party measuring instrument, said connection assembly comprising printed circuit boards, a plug, and an enclosure; said device provides continuous, uninterrupted monitoring of at least one patient parameter; said device is capable of communicating with a bedside monitor; said device is worn by and remains with an assigned patient for the duration of care; and, said device is designed in a compact and lightweight manner to enhance portability.

16. The portable, self-contained device used to monitor patient parameters of claim 17, further comprising an integrated touchscreen display, alarm annunciation, and limited data storage capability.

17. A connection assembly used to translate electronic information from a first protocol to a second protocol, comprising:
a. a plug at a first end of said assembly for connecting a first device cable from a first device, said first device operating on said first protocol;
b. at least one first connector at a second end of said assembly opposite said first end of said assembly, for connecting at least one printed circuit board;
c. at least one second connector at said second end of said assembly opposite said first end of said assembly, for connecting at least one second device cable from a second device, said second device operating on said second protocol;
d. at least one printed circuit board capable of translating said electronic information from said first protocol to said second protocol, said printed circuit board connected to said at least one first connector at said second end of said assembly; and,
e. at least one movable elongate member attached to said plug along an exterior edge of said plug, said at least one movable elongate member movable from a first position to a second position wherein said movable elongate member forms an enclosure at least partially encompassing said at least one printed circuit board and said second device cable when said movable elongate member is in said second position.

18. The connection assembly used to translate electronic information from a first protocol to a second protocol of claim 17, wherein said plug includes a gasket around the outer edge of said plug to prevent liquid ingress into the assembly.

19. A system for providing continuous patient monitoring, comprising:
a. a portable, self-contained device used to monitor patient parameters, comprising an internal battery, at least one connection for a parameter measuring instrument, and at least one connection assembly for connection of a third party measuring instrument, said connection assembly comprising printed circuit boards, a plug, and an enclosure, wherein; said device provides continuous, uninterrupted monitoring of at least one patient parameter; said device is capable of communicating with a bedside monitor and a patient monitoring network; said device is worn by and remains with an assigned patient for the duration of care; and, said device is designed in a compact and lightweight manner to enhance portability;
b. at least one patient parameter measuring instrument connected to said device;
c. a bedside monitor in communication with said device;
d. at least one external display connected to said bedside monitor; and,
e. a hospital network in communication with said bedside monitor.

20. The system for providing continuous patient monitoring of claim 19, wherein said device includes an integrated wireless network card and said device is in direct communication with said hospital network.
